(19) United States
(12) Patent Application Publication
(10) Pub. No.: US 2005/0288675 A1
(43) Pub. Date: Dec. 29, 2005

(54) Disk incision repair method

(75) Inventors: Daniel Payne Robertson, Ocala, FL (US); David W. Szalay, Germantown, TN (US)

Correspondence Address: PEARL COHEN ZEDEK, LLP, 10 ROCKEFELLER PLAZA, SUITE 1001, NEW YORK, NY, 10020, US

(73) Assignee: Med Ideas, LLC

(21) Appl. No.: 11/156,457

(22) Filed: Jun. 21, 2005

Related U.S. Application Data
(60) Provisional application No. 60/582,081, filed on Jun. 24, 2004.

(51) Int. Cl. A61B 17/56

(52) U.S. Cl. 606/75

(57) ABSTRACT

The present invention provides methods of implementing inter-vertebral disk surgery, including methods of sealing an inter-vertebral disk incision, preventing or inhibiting herniation, and/or stimulating or facilitating healing of an inter-vertebral disk. Also provided are annulus closure tools.

### CROSS REFERENCE TO RELATED APPLICATIONS

This application claims priority of U.S. Provisional Application Ser. No. 60/582,081, filed Jun. 24, 2004. This application is hereby incorporated in its entirety by reference herein

### FIELD OF THE INVENTION

The present invention provides methods of implementing inter-vertebral disk surgery, including methods of sealing an inter-vertebral disk incision, preventing or inhibiting herniation, and/or stimulating or facilitating healing of an inter-vertebral disk. Also provided are annulus closure tools.

### BACKGROUND OF THE INVENTION

Back pain is the leading cause of healthcare expenditures in the US. The worldwide back pain market is valued at more than $2 billion and is expanding at over 25% annually. The market segment growing most rapidly is inter-vertebral disc replacement and repair, expected to exceed $1 billion by 2007.

The vertebrae of the spinal column bear the majority of the weight placed on the spine, and additionally protect the spinal cord. As can be seen with reference to FIGS. 1 and 2, inter-vertebral discs are flat, round, cushions that lie between each vertebrae and absorb shock and other types of stresses. The discs have numerous functions, including maintaining separation between adjacent vertebral bodies and facilitating motion.

Each inter-vertebral disc is comprised of two main elements: a strong outer ring called the annulus fibrosus, and a soft center called the nucleus pulposus. The disc annulus is a complex structure of highly organized collagen fibrils and the strongest layer of the disc. The disc annulus surrounds the inner nucleus pulposus, which has a more random collagen organization and proteoglycans.

FIG. 3 depicts a number of disc ailments that commonly occur. Degenerative discs are characterized by circumferential tears or fissures in the outer annulus. Bulging disks and herniated disks are characterized by localized distension of the annulus. Thinning disks result from loss of nucleus pulposus mass.

A number of strategies are currently used to repair disc ailments. In discectomy, the wall of the annulus is opened to remove a herniated disc. In artificial nucleus replacement, the nucleus pulposus (“nucleus”) is replaced with a prosthetic nucleus. Typically, an incision is made through the disc annulus to expose the nucleus, which is then removed, and dehydrated artificial nucleus implants are inserted into the cavity.

Both discectomy and artificial nucleus replacement require an effective method for sealing the incision after surgery, in order to decrease the number of disc re-herniations, preserve disc structure and retain motion segment and stability. Unsealed incisions leave an opening in the disc, which weakens the disc structure. Re-herniation through the incised annulus occurs in a significant percentage of cases (up to 30%), with 14% requiring a second operation. Presently, spinal surgeons do not have an effective method or device to repair an annulus.

### SUMMARY OF THE INVENTION

Embodiments of the present invention provide methods of sealing an inter-vertebral disk incision, preventing or inhibiting herniation, and/or stimulating or facilitating healing of an inter-vertebral disk, using an annulus closure tool.

In one embodiment, the present invention provides a method of sealing an inter-vertebral disk incision, comprising applying an annulus closure tool to an inter-vertebral disk incision, the annulus closure tool comprising a first leg, a second leg, and a connection member connecting the first leg to the second leg, the first leg and the second leg comprising projections protruding therefrom, thereby sealing an inter-vertebral disk incision.

In another embodiment, the present invention provides a method of facilitating healing of an inter-vertebral disk incision, comprising applying an annulus closure tool to the inter-vertebral disk incision, the annulus closure tool comprising a first leg, a second leg, and a connection member connecting the first leg to the second leg, the first leg and the second leg comprising projections protruding therefrom, thereby facilitating healing of an inter-vertebral disk incision.

In another embodiment, the present invention provides a method of inhibiting extrusion or re-herniation of a nucleus pulposus from an inter-vertebral disk, wherein the inter-vertebral disk has a tear, fissure, or incision, comprising applying an annulus closure tool to an inter-vertebral disk incision, the annulus closure tool comprising a first leg, a second leg, and a connection member connecting the first leg to the second leg, the first leg and the second leg comprising projections protruding therefrom, thereby inhibiting extrusion or re-herniation of a nucleus pulposus from an inter-vertebral disk.

In another embodiment, the present invention provides a method of inhibiting extrusion or re-herniation of a nucleus replacement device or nucleus replacement material from an inter-vertebral disk, comprising applying an annulus closure tool to an inter-vertebral disk incision, the annulus closure tool comprising a first leg, a second leg, and a connection member connecting the first leg to the second leg, the first leg and the second leg comprising projections protruding therefrom, thereby inhibiting extrusion or re-herniation of a nucleus replacement device or nucleus replacement material from an inter-vertebral disk.

### BRIEF DESCRIPTION OF THE DRAWINGS

The principles and operation of the system, apparatus, and method according to the present invention may be better understood with reference to the drawings, and the following description, it being understood that these drawings are given for illustrative purposes only and are not meant to be limiting, wherein:

FIG. 1 illustrates an invertebral disc anatomy.

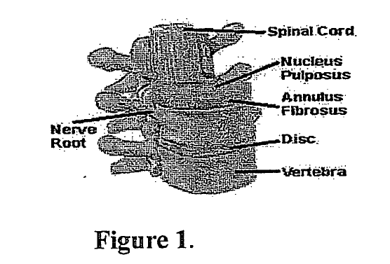

FIG. 2 illustrates a side view of vertebral motion.

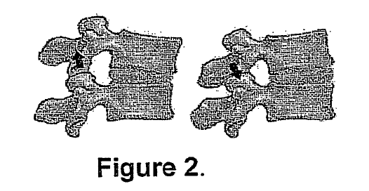

FIG. 3 illustrates an anatomical depiction of various disk ailments.

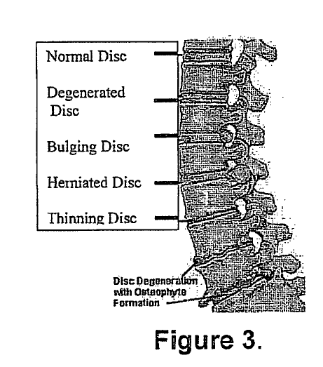

FIG. 4 illustrates degenerative changes seen in a section of porcine inter-vertebral disc in which an incision was made in the annulus.

FIGS. 5A-B illustrate the histology of a degenerative disc model.

FIG. 6 illustrates proliferation in annular cells from an incised inter-vertebral disk, as assessed by 5-bromo-2-deoxyuridine (BrdU) staining.

FIG. 7 illustrates the experimental setup for the biomechanical testing of the annulus closure tools.

FIG. 8 illustrates an annulus closure tool that has undergone expulsion of one end of the staple from a disk in which it was inserted.

It will be appreciated that for simplicity and clarity of illustration, elements shown in the drawings have not necessarily been drawn to scale. For example, the dimensions of some of the elements may be exaggerated relative to other elements for clarity. Further, where considered appropriate, reference numerals may be repeated among the drawings to indicate corresponding or analogous elements throughout the serial views.

### DETAILED DESCRIPTION OF THE PRESENT INVENTION

The following description is presented to enable one of ordinary skill in the art to make and use the invention as provided in the context of a particular application and its requirements. Various modifications to the described embodiments will be apparent to those with skill in the art, and the general principles defined herein may be applied to other embodiments. Therefore, the present invention is not intended to be limited to the particular embodiments shown and described, but is to be accorded the widest scope consistent with the principles and novel features herein disclosed. In other instances, well-known methods, procedures, and components have not been described in detail so as not to obscure the present invention.

Embodiments of the present invention provide methods of sealing an inter-vertebral disk incision, preventing or inhibiting herniation, and/or stimulating or facilitating healing of an inter-vertebral disk, using an annulus closure tool.

In one embodiment, the present invention provides a method of sealing an inter-vertebral disk incision, comprising applying an annulus closure tool to an inter-vertebral disk incision, the annulus closure tool comprising a first leg, a second leg, and a connection member connecting the first leg to the second leg, the first leg and the second leg comprising projections protruding therefrom, thereby sealing an inter-vertebral disk incision.

In another embodiment, the present invention provides a method of facilitating healing of an inter-vertebral disk incision, comprising applying an annulus closure tool to the inter-vertebral disk incision, the annulus closure tool comprising a first leg, a second leg, and a connection member connecting the first leg to the second leg, the first leg and the second leg comprising projections protruding therefrom, thereby facilitating healing of an inter-vertebral disk incision

In another embodiment, the present invention provides a method of inhibiting extrusion of a nucleus pulposus from an inter-vertebral disk, wherein the inter-vertebral disk has a tear, fissure, or incision, comprising applying an annulus closure tool to an inter-vertebral disk incision, the annulus closure tool comprising a first leg, a second leg, and a connection member connecting the first leg to the second leg, the first leg and the second leg comprising projections protruding therefrom, thereby inhibiting extrusion of a nucleus pulposus from an inter-vertebral disk.

In another embodiment, the present invention provides a method of inhibiting re-herniation of a nucleus pulposus from an inter-vertebral disk, wherein the inter-vertebral disk has a tear, fissure, or incision, comprising applying an annulus closure tool to an inter-vertebral disk incision, the annulus closure tool comprising a first leg, a second leg, and a connection member connecting the first leg to the second leg, the first leg and the second leg comprising projections protruding therefrom, thereby inhibiting re-herniation of a nucleus pulposus from an inter-vertebral disk.

As provided herein, the results of the present invention demonstrate that the annulus closure tool of the present invention can be inserted into and remain embedded in calf and pig inter-vertebral disks following significant and prolonged mechanical stress. Further provided herein are methods of testing the methods and compositions of the present invention, as demonstrated in the Examples herein. Thus, in another embodiment, the present invention provides methods and compositions that are optimized based on the results of testing methods disclosed herein. In another embodiment, the length of the connection member and/or the legs is modified to perform minimally invasive spine surgery (MIS spine surgery). In another embodiment, the length of the connection member and or the legs remains unmodified for performing MIS spine surgery. Each possibility represents a separate embodiment of the present invention.

The disc shape and height of the pig spine is very similar to the human spine; thus, the pig spine is a good model for methods of treating incisions and fissures in the human spine. Some difference does exist in the thickness of the lamina and pars articularis between pig and human spines, which resulted in restricted access to the posterior spine in the experiments performed with pig spines. This, however, did not affect the ease of implantation, once the bony tissues were adequately prepared, demonstrating that methods of the present invention have utility in human spines.

Thus, methods of the present invention have utility in sealing an inter-vertebral disk incision, preventing or inhibiting herniation, and stimulating or facilitating healing of an inter-vertebral disk.

In another embodiment, the present invention provides a method of inhibiting extrusion of a nucleus replacement device from an inter-vertebral disk, comprising applying an annulus closure tool to an inter-vertebral disk incision, the annulus closure tool comprising a first leg, a second leg, and a connection member connecting the first leg to the second leg, the first leg and the second leg comprising projections protruding therefrom, thereby inhibiting extrusion of a nucleus replacement device from an inter-vertebral disk.

In another embodiment, the present invention provides a method of inhibiting re-herniation of a nucleus replacement device from an inter-vertebral disk, comprising applying an annulus closure tool to an inter-vertebral disk incision, the annulus closure tool comprising a first leg, a second leg, and a connection member connecting the first leg to the second leg, the first leg and the second leg comprising projections protruding therefrom, thereby inhibiting re-herniation of a nucleus replacement device from an inter-vertebral disk.

In another embodiment, the present invention provides a method of inhibiting extrusion of a nucleus replacement material from an inter-vertebral disk, comprising applying an annulus closure tool to an inter-vertebral disk incision, the annulus closure tool comprising a first leg, a second leg, and a connection member connecting the first leg to the second leg, the first leg and the second leg comprising projections protruding therefrom, thereby inhibiting extrusion of a nucleus replacement material from an inter-vertebral disk.

In another embodiment, the present invention provides a method of inhibiting re-herniation of a nucleus replacement material from an inter-vertebral disk, comprising applying an annulus closure tool to an inter-vertebral disk incision, the annulus closure tool comprising a first leg, a second leg, and a connection member connecting the first leg to the second leg, the first leg and the second leg comprising projections protruding therefrom, thereby inhibiting re-herniation of a nucleus replacement material from an inter-vertebral disk.

In one embodiment, the above methods of inhibiting extrusion or re-herniation of a nucleus replacement device or material from an inter-vertebral disk are performed following disc replacement surgery. Disc replacement surgery is a known technique for treating disk ailments involving loss of nucleus pulposus mass, e.g. thinning disks, and involves introduction of a nucleus replacement device or material into the disk. Extrusion of the device or material from the disk is a post-operative complication that prevents long-term resolution of the ailment.

In one embodiment, the inter-vertebral disk incision that is healed, closed or treated by methods of the present invention is a slit incision. In another embodiment, the incision is any other type of surgical incision known in the art. In another embodiment, the incision is a non-surgical incision. Each possibility represents a separate embodiment of the present invention.

In another embodiment, the present invention provides a method of repairing a bulging or herniated inter-vertebral disk, comprising applying an annulus closure tool to a site of bulging or herniation in the inter-vertebral disk, the annulus closure tool comprising a first leg, a second leg, and a connection member connecting the first leg to the second leg, the first leg and the second leg comprising projections protruding therefrom, thereby repairing a bulging or herniated inter-vertebral disk.

In another embodiment, the present invention provides an annulus closure tool comprising a first leg, a second leg, and a connection member connecting the first leg to the second leg, wherein the length of the connection member is between about 5 and about 25 mm.

In another embodiment, the length of the connection member of the annulus closure tool of the present invention is between about 5 and about 25 mm. In another embodiment, the length is between about 3-20 mm. In another embodiment, the length is between about 5-18 mm. In another embodiment, the length is between about 6-17 mm. In another embodiment, the length is between about 7-16 mm. In another embodiment, the length is between about 8-15 mm. In another embodiment, the length is between about 9-14 mm. In another embodiment, the length is between about 10-13 mm. In another embodiment, the length is between about 10.5-12.5 mm. In another embodiment, the length is between about 11-12 mm.

In another embodiment, the length of the connection member is about 6 mm. In another embodiment, the length is about 7 mm. In another embodiment, the length is about 8 mm. In another embodiment, the length is about 9 mm. In another embodiment, the length is about 10 mm. In another embodiment, the length is about 11 mm. In another embodiment, the length is about 12 mm. In another embodiment, the length is about 13 mm. In another embodiment, the length is about 14 mm. In another embodiment, the length is about 15 mm. In another embodiment, the length is about 16 mm. In another embodiment, the length is about 17 mm. In another embodiment, the length is about 18 mm. In another embodiment, the length is about 19 mm. In another embodiment, the length is about 20 mm. In another embodiment, the length is about 21 mm. In another embodiment, the length is about 22 mm. In another embodiment, the length is about 24 mm. In another embodiment, the length is about 25 mm. In another embodiment, the length is about 23 mm.

In another embodiment, the present invention provides an annulus closure tool comprising a first leg, a second leg, and a connection member connecting the first leg to the second leg, wherein the length of the first leg and the second leg is between about 5 and about 15 mm.

In another embodiment, the length of the legs is between about 6-15 mm. In another embodiment, the length is between about 7-15 mm. In another embodiment, the length is between about 8-14 mm. In another embodiment, the length is between about 9-13 mm. In another embodiment, the length is between about 10-12 mm. In another embodiment, the length is between about 10.5-11.5 mm.

In another embodiment, the length of the legs is about 6 mm. In another embodiment, the length is about 7 mm. In another embodiment, the length is about 8 mm. In another embodiment, the length is about 9 mm. In another embodiment, the length is about 10 mm. In another embodiment, the length is about 11 mm. In another embodiment, the length is about 12 mm. In another embodiment, the length is about 13 mm. In another embodiment, the length is about 14 mm. In another embodiment, the length is about 15 mm. In another embodiment, the length is about 16 mm.

Each length of the legs and/or connection member of the annulus closure tool represents a separate embodiment of the present invention.

In one embodiment, the connection member of the annulus closure tool of methods and compositions of the present invention is a convergent connection member. In another embodiment, a non-convergent connection member is utilized. In another embodiment, the convergent connection member stimulates healing of the incision by providing a convergent pressure across the incision. Each possibility represents a separate embodiment of the present invention

In one embodiment, the annulus closure tool of the present invention comprises projections or spikes. In another embodiment, the annulus closure tool does not comprise projections or spikes. “Projections,” in one embodiment, refers to spikes that protrude from the legs of a tool such as an annulus closure tool or a surgical staple. In another embodiment, “projections” refers to blunt pegs that protrude therefrom. In another embodiment, “projections” refers to any other type of projection known in the art to function in anchoring a surgical staple or suture into tissue. Each possibility represents a separate embodiment of the present invention.

In one embodiment, the projections or spikes enable the closure tool to be better secured within the inter-vertebral discs. In another embodiment, the projections or spikes protrude from the legs of the annulus closure tool. In another embodiment, the projections or spikes protrude from the connection member. Each possibility represents a separate embodiment of the present invention.

In one embodiment, the length of the projections or spikes is between about 0.5 to 2.0 mm in length. In another embodiment, the length is between about 0.6 and about 1.9 mm. In another embodiment, the length is between about 0.7 to 1.8 mm. In another embodiment, the length is between about 0.8 to 1.7 mm. In one embodiment, the length is between about 0.9 to 1.6 mm. In another embodiment, the length is between about 1.0 to 1.5 mm. In another embodiment, the length is between about 1.1 to 1.4 mm. In another embodiment, the length is between about 1.15 to 1.35 mm. In another embodiment, the length is between about 1.2 to 1.25 mm.

In another embodiment, the length is about 0.6 mm. In another embodiment, the length is about 0.7 mm. In another embodiment, the length is about 0.8 mm. In another embodiment, the length is about 0.9 mm. In another embodiment, the length is about 1.0 mm. In another embodiment, the length is about 1.1 mm. In another embodiment, the length is about 1.2 mm. In another embodiment, the length is about 1.3 mm. In another embodiment, the length is about 1.4 mm. In another embodiment, the length is about 1.5 mm. Each length represents a separate embodiment of the present invention.

In one embodiment, the projections or spikes vary in length. In another embodiment, the projections or spikes have a uniform length. In another embodiment, the projections or spikes have a substantially uniform length. Each possibility represents a separate embodiment of the present invention.

In another embodiment, the projections or spikes are between about 0.5 to 2.0 mm in diameter. In another embodiment, the diameter is between about 0.6 to 10.7 mm. In another embodiment, the diameter is between about 0.7 to 1.8 mm. In another embodiment, the diameter is between about 0.8 to 1.7 mm. In one embodiment, the diameter is between about 0.9 to 1.6 mm. In another embodiment, the diameter is between about 1.0 to 1.5 mm. In one embodiment, the diameter is between about 1.1 to 1.4 mm. In another embodiment, the diameter is between about 1.15 to 1.35 mm. In another embodiment, the diameter is between about 1.2 to 1.25 mm

In another embodiment, the diameter is about 0.6 mm. In another embodiment, the diameter is about 0.7 mm. In another embodiment, the diameter is about 0.8 mm. In another embodiment, the diameter is about 0.9 mm. In another embodiment, the diameter is about 1.0 mm. In another embodiment, the diameter is about 1.1 mm. In another embodiment, the diameter is about 1.2 mm. In another embodiment, the diameter is about 1.3 mm. In another embodiment, the diameter is about 11.4 mm. In another embodiment, the diameter is about 1.5 mm. Each diameter represents a separate embodiment of the present invention.

In one embodiment, the projections or spikes vary in diameter. In another embodiment, the projections or spikes have a uniform diameter. In another embodiment, the projections or spikes have a substantially uniform diameter. Each possibility represents a separate embodiment of the present invention.

In one embodiment, the projections or spikes protrude at an angle of between about 15-90 degrees relative to the axis of the legs. In another embodiment, the angle is between about 25-80 degrees relative to the axis of the legs. In another embodiment, the angle is between about 30-75 degrees. In another embodiment, the angle is between about 35-70 degrees. In another embodiment, the angle is between about 40-65 degrees. In another embodiment, the angle is between about 45-60 degrees. In another embodiment, the angle is between about 50-55 degrees. In another embodiment, the angle is between about 35-50 degrees. In another embodiment, the angle is between about 55-70 degrees. In another embodiment, the angle is between about 60-75 degrees. In another embodiment, the angle is between about 65-80 degrees. In another embodiment, the angle is between about 70-85 degrees. In another embodiment, the angle is between about 75-90 degrees.

In another embodiment, the angle is about 30 degrees. In another embodiment, the angle is about 35 degrees. In another embodiment, the angle is about 40 degrees. In another embodiment, the angle is about 45 degrees. In another embodiment, the angle is about 50 degrees. In another embodiment, the angle is about 55 degrees. In another embodiment, the angle is about 60 degrees. In another embodiment, the angle is about 65 degrees. In another embodiment, the angle is about 70 degrees. In another embodiment, the angle is about 75 degrees. In another embodiment, the angle is about 80 degrees. In another embodiment, the angle is about 85 degrees. In another embodiment, the angle is about 90 degrees. Each angle represents a separate embodiment of the present invention.

In one embodiment, the projections or spikes vary in the angle at which they protrude relative to the axis of the legs. In another embodiment, the angle is uniform among the projections or spikes. In another embodiment, the angle is substantially uniform among the projections or spikes. Each possibility represents a separate embodiment of the present invention.

Each type of projection, spike, or peg of the annulus closure tool represents a separate embodiment of the present invention.

In another embodiment, the legs, connection member, and/or projections of the annulus closure tool of the present invention comprises a bioresorbable material. In another embodiment, the legs, connection member, and/or projections is composed of a bioresorbable material. In one embodiment, the bioresorbable material is a polymer of lactide. In another embodiment, the material is a polymer of glycolide. In another embodiment, the material is a lactide/glycolide copolymer. In another embodiment, the material is poly-dioxanone. In another embodiment, the material is trimethylene carbonate. In another embodiment, the material is polyethylene oxide. In another embodiment, the material is poly (epsilon-caprolactone). In another embodiment, the material is levolactic acid. In another embodiment, the material is poly(lactic acid). In another embodiment the material is Polylactide®. In another embodiment, the material is any other bioresorbable material known in the art that is suitable for use in a surgical staple or suture. In another embodiment, the material is a blend of two or more of the above materials. Each possibility represents a separate embodiment of the present invention.

In another embodiment, the annulus closure tool comprises a non-bioresorbable material. In another embodiment, the annulus closure tool is composed of a non-bioresorbable material. In one embodiment, the non-bioresorbable material is nitonal. In another embodiment, the material is titanium. In another embodiment, the material is polyethylene. In another embodiment, the material is polyetheretherketone (PEEK). In another embodiment, the material is any other non-bioresorbable material known in the art that is suitable for use in a surgical staple or suture. Each possibility represents a separate embodiment of the present invention

In another embodiment, the connection member of the annulus closure tool of methods and compositions of the present invention comprises a flexible material. In another embodiment, the connection member is composed of a flexible material. In one embodiment, the flexible material is a lactide/glycolide copolymer. In another embodiment, the flexible material is one of the above materials mentioned for inclusion in the annulus closure tool. Each material represents a separate embodiment of the present invention.

In another embodiment, the connection member comprises a suture. In another embodiment, the connection member consists of a suture. Each possibility represents a separate embodiment of the present invention.

In one embodiment, a pledget is attached to or associated with a connection member of the annulus closure tool of methods and compositions of the present invention. In one embodiment, “pledget” refers to an absorbent pad. In another embodiment, the pledget is any other type of pledget known in the art. In another embodiment, the pledget is used to medicate, drain, or protect a wound or sore. In another embodiment, the pledget is flat. The use of surgical pledgets is well known in the art, and is described, for example, in U.S. Pat. Nos. 5,733,308 and 4,823,794. Each type of pledget known in the art represents a separate embodiment of the present invention.

In another embodiment, the pledget is associated with one or more of the projections, spikes, or pegs of the annulus closure tool of methods and compositions of the present invention.

In another embodiment, the pledget is associated with one or more of the legs of the annulus closure tool of methods and compositions of the present invention.

In another embodiment, the annulus closure tool of methods and compositions of the present invention further comprises a growth factor, healing factor, or healing device. The use of growth factors, healing factors, and healing devices are well known in the art, and is described, for example, in U.S. Pat. Nos. 5,703,047 and 6,810,288, and in Ichioka S et al, J Wound Care 14(3):105-9, 2005. Each type of growth factor, healing factor, and healing device known in the art represents a separate embodiment of the present invention.

In another embodiment, a growth factor is associated with the pledget of methods and compositions of the present invention. In another embodiment, a healing factor is associated with the pledget. In another embodiment, a healing device is associated with the pledget.

In another embodiment, a growth factor is associated with the first leg and/or the second leg of the annulus closure device. In another embodiment, a healing factor is associated with the first leg and/or the second leg of the annulus closure device. In another embodiment, a healing device is associated with the first leg and/or the second leg of the annulus closure device.

In another embodiment, a growth factor is associated with the connection member of the annulus closure device. In another embodiment, a healing factor is associated with the connection member of the annulus closure device. In another embodiment, a healing device is associated with the connection member of the annulus closure device.

In another embodiment, a growth factor is associated with one or more of the projections of the annulus closure device. In another embodiment, a healing factor is associated with one or more of the projections of the annulus closure device. In another embodiment, a healing device is associated with one or more of the projections of the annulus closure device.

In one embodiment, the annulus closure tool of methods and compositions of the present invention comprises a blunt peg at the bottom of one or more of its legs. In another embodiment, the blunt peg may be associated with a delivery mechanism for a growth factor or device or a healing factor or device. Each possibility represents a separate embodiment of the present invention.

In another embodiment, a device or vehicle containing a healing device, healing factor or growth factor is implanted within the annulus closure tool of methods and compositions of the present invention. In one embodiment, the device is implanted within a leg of the tool. In another embodiment, the device is implanted within the connection member of the tool. In another embodiment, the device is implanted within a spike or projection of the tool. Each possibility represents a separate embodiment of the present invention.

Each of the above types of attaching a growth factor, healing factor, or healing devices to an annulus closure tool represents a separate embodiment of the present invention.

According to some embodiments of the present invention, a method of the present invention includes, following a surgical procedure, applying the annulus closure tool or a similar tool to the annulus incision to be closed. Application of the annulus closure tool assists, in this embodiment, in preventing or inhibiting herniation of an inter-vertebral disk following a surgical procedure. In another embodiment, any combination of the steps described herein may be implemented. In another embodiment, other steps or series of steps, in addition to those described herein, are used. In some embodiments, a method of the present invention is used to stimulate or facilitate healing of an inter-vertebral disk incision.

In another embodiment, the annulus closure tool of methods and compositions of the present invention is delivered or applied to the disk using an annulus closure tool delivery device. In another embodiment, the annulus closure tool is delivered or applied using any other method known in the art of delivering or applying a surgical staple.

Annulus closure tool delivery devices are well known in the art—see, e.g, U.S. Pat. Nos. 6,446,854 and 6,312,447. In one embodiment, the annulus closure tool delivery device is oriented at 90 degrees relative to the annulus wall. In another embodiment, the annulus closure tool delivery device is oriented at a different angle relative to the annulus wall. In another embodiment, pressure is applied to the annulus closure tool delivery device before firing. In another embodiment, the handle of the annulus closure tool delivery device is modified to enhance application of the annulus closure tool or to improve ergonomics. In another embodiment, the annulus closure tool delivery device includes a plurality of preloaded annulus closure tools. The annulus closure tool delivery device enables, in some embodiments, the annulus closure tool to be precisely entered into a selected area of an inter-vertebral disk. In another embodiment, delivery device enables the delivery of an annulus closure tool at a selected pressure, and provides slight deflection at the incision point, resulting in convergence and closure of the incision

In one embodiment, the annulus closure tool delivery device comprises internal “rails” to guide the application of the annulus closure tool, as described, for example, in U.S. Pat. Nos. 6,446,854 and 6,312,447. In one embodiment, the rails of the annulus closure tool delivery device are straight and oriented parallel to the axis of the annulus closure tool delivery device, in order to deliver the annulus closure tool at an angle parallel to the axis of the annulus closure tool delivery device. In another embodiment, the rails of the annulus closure tool delivery device are curved or oriented at an angle other than parallel to the axis of the annulus closure tool delivery device, in order to deliver the annulus closure tool delivery device at an angle other than parallel to the annulus closure tool delivery device.

Each type of annulus closure tool delivery device represents a separate embodiment of the present invention.

In another embodiment, the present invention provides a kit comprising a reagent utilized in performing a method of the present invention. In another embodiment, the present invention provides a kit comprising a composition, tool, or instrument of the present invention.

The foregoing description of the embodiments of the invention has been presented for the purposes of illustration and description. It is not intended to be exhaustive or to limit the invention to the precise form disclosed. It should be appreciated by persons skilled in the art that many modifications, variations, substitutions, changes, and equivalents are possible in light of the above teaching. It is, therefore, to be understood that the appended claims are intended to cover all such modifications and changes as fall within the true spirit of the invention.

### EXPERIMENTAL DETAILS SECTION

### Example 1

### Biomechanical Testing of Annulus Closure Tools

### Materials and Experimental Methods

Storage of Samples

Fresh-frozen calf spines were sealed and stored at −20° C. These specimens were potted for 2-3 hr in an aluminum-containing epoxy, which helps to heat-sink the polymerization energy away from the spine. Samples were greased within ordinary bearing grease and covered with plastic bags as they cured in the pot fixtures to minimize moisture loss.

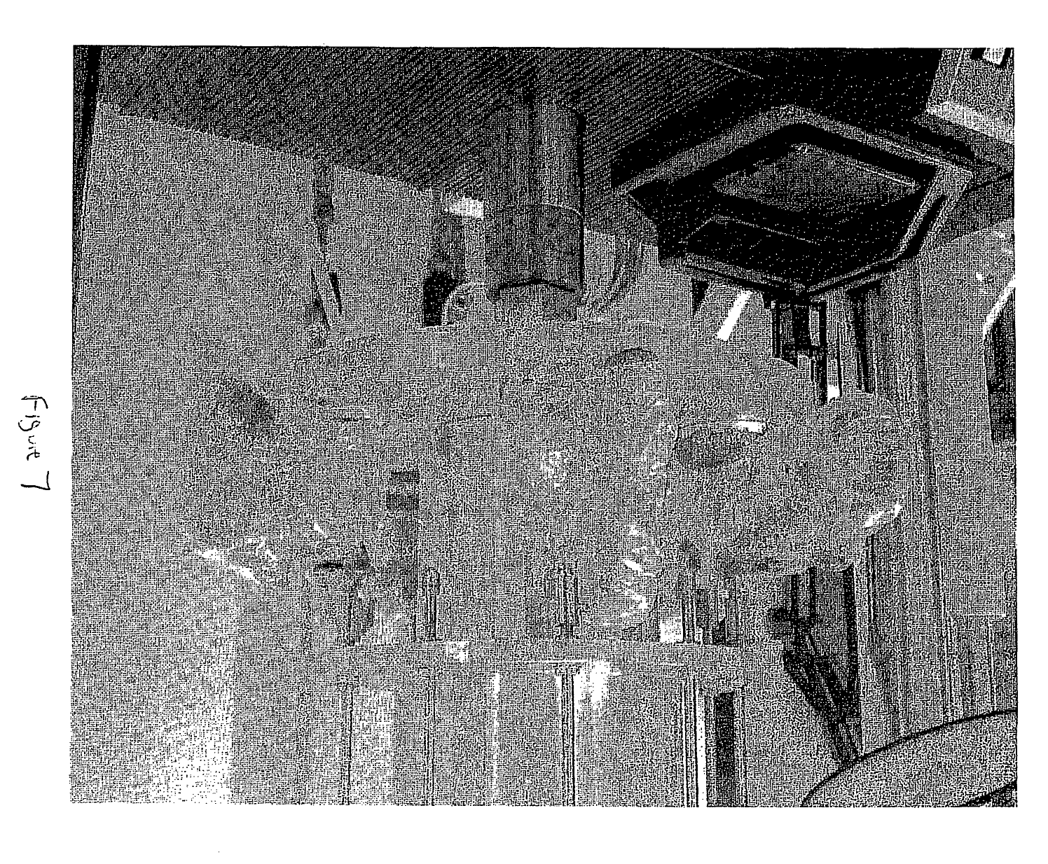

The following morning, specimens were placed in the test fixture and wrapped with Saran® wrap (FIG. 7) further trap the moisture. There was no visible change in the quality of the discs over the duration of the tests.

Never-frozen pig spines were sprayed periodically with cold saline to maintain their hydrated salt balance and temperature. The specimens warmed up to room temperature as they went through the potting process, which utilized a relatively fast-curing epoxy called marine chocking, from Loctite. In this case, the curing process took less than 1 hr. For the low-load specimens, cure time was considerably less, as the amount of potting material was less than ⅓ of that of the high-load specimens.

### Results

Annulus closure tools of the present invention were implanted into 5 fresh-frozen calf spines. The closure tools were V-shaped, with legs 10 millimeter (mm) in length that had barbs protruding therefrom to anchor them in the tissue, connected by a 4 mm long braided connection member composed of a resorbable copolymer, a polyester derivative of lactic acid and glycolic acids.

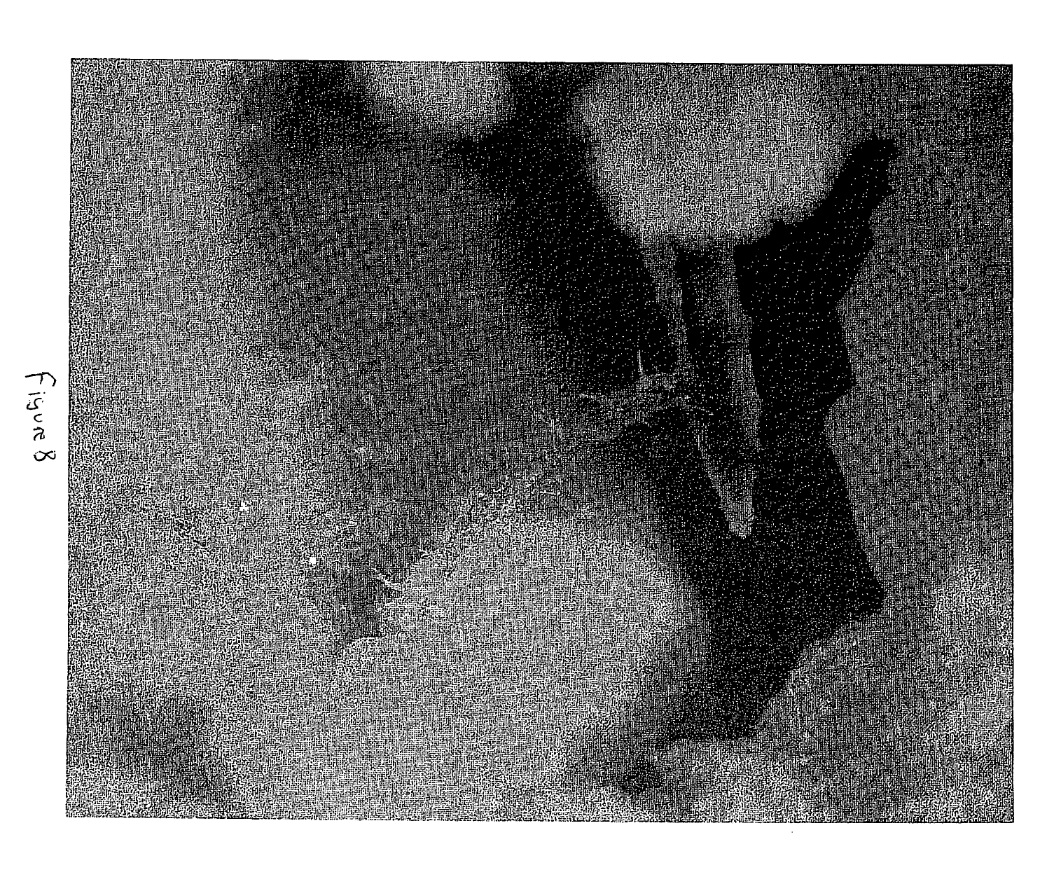

Retention of the closure tools in the first 10 spines was tested using a servo-hydraulic Instron 8501 Material Testing System® (Instron Corp, Canton, Mass.). The crosshead of the test fixture was attached to the connection member, and force was applied parallel to the axis of the spine at 2 Hz, cycling between 40 and 160 pounds of force, for 34,000 cycles each over the course of 5 hours (hr). Little or no deformation of the disks was noticeable to the eye during the testing. Only 1 of the specimens experienced expulsion (of one end of the staple) from the disc tissues (see FIG. 8 for a representative diagram). In addition, the specimens exhibited flexion bending of 1-2 degrees during testing.

In addition, 5 never-frozen pig spines were tested using the above apparatus. Pigs were sacrificed on day 0, spines were harvested on day one, and testing was performed in day 2. In this case, the apparatus was run for 150,000 total cycles over 22 hr. None of these specimens showed expulsion. The tissue did not appear decayed, as extended measures were taken to assure their freshness and hydration (see Materials and Experimental Methods section). As in the previous set of specimens, these generally exhibited 1-2 degrees of flexion bending during testing.

Three additional never-frozen pig spines were subjected to high-load cyclic testing, with a significant component of flexion bending, using an electromechanical Instron 4502 Materials Testing Machine®. 5 Hz cyclic loading of between 10 and 300 pounds for about 10,000 cycles was applied to the spines over 35 min. During this time, the specimens flexed progressively, due to viscoelastic give, reaching a maximum of about 10 degrees of flexion. At this point, the minimum flexion value at the low-load end of the cycle was between 8-9 degrees. Thus, the amount of flexion induced in the specimen in each cycle did not change appreciably; rather, the offset flexion angle increased. To avoid failure of the ligamentous spine itself, the point of load application was reset slightly backwards toward the center of rotation to reduce the amount of flexion. The cyclic loading was continued for another 1.5-2 hr, totaling approximately 40,000 cycles. Only 1 of the 3 specimens exhibited expulsion (of one end of the staple) under these conditions.

The results presented in this Example demonstrate that annulus closure tools of the present invention are able to remain embedded in inter-vertebral disks following significant and prolonged mechanical stress. Thus, methods of the present invention have utility in sealing an inter-vertebral disk incision, preventing or inhibiting herniation, and stimulating or facilitating healing of an inter-vertebral disk.

### Example 2

### Optimization of Annulus Closure Tool

Parameters of the annulus closure tool of the present invention (e.g. length of the staple legs; length of the connection member; material composition of the connection member; material composition of the staple legs; shape, number, and composition of the protrusions from the legs; and pressure, angle and deflection of delivery of the annulus closure tool) are varied in order to improve ability of the tool to remain embedded in vertebral disks following mechanical stress. Resistance of the embedded tools to expulsion from the tissues is tested using the methods described in Example 1. In addition, other parameters (ease of insertion, reproducibility, etc.) are tested for the modified tools. These experiments result in development of annulus closure tools that are improved in the above parameters.

### Example 3

### Use of the Annulus Closure Tool Improves Healing of Annular Incisions

Inter-vertebral discs of pigs are incised using a standard scalpel used to create portals for arthroscopic surgery, after which the disks are subjected to no further treatment (Group A), or closure using an annulus closure tool of the present invention (Group B) Post-operative ability of the animals to ambulate is assessed to measure neurological impairment, an indicator of spinal cord damage. Animals are euthanized by anesthetic overdose at 4, 8 or 12 weeks after surgery.

Following sacrifice, disks are examined In addition, the length of the annular tear is measured and pressure-volume testing, a reliable measure of annular strength (Panjabi M M et al, Spine 13: 913-917, 1998), is performed to assess the degree of healing. Annulus closure tools of the present invention significantly improve healing of annular incisions

### Example 4

### Use of the Annulus Closure Tool Descreases Inter-Vertebral Disc Degeneration Resulting from an Incision

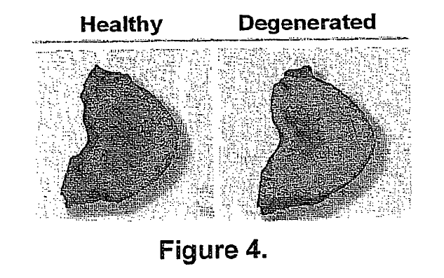

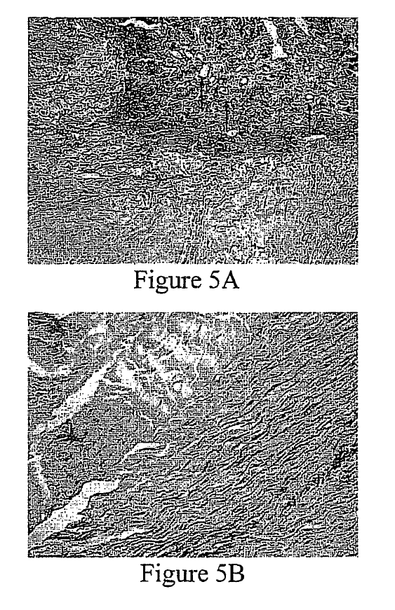

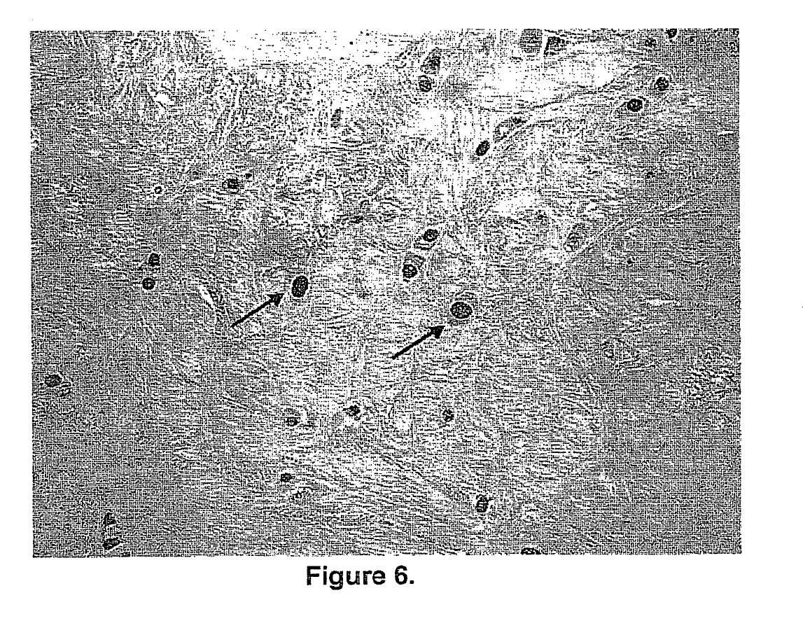

The ability of methods of the present invention to prevent inter-vertebral disc degeneration is tested using a model of inter-vertebral disc degeneration described in FIGS. 4-6. FIG. 4 depicts degenerative changes seen in a section of porcine inter-vertebral disc in which an incision was made in the annulus. FIGS. 5 A-B depict the histology of a Degenerative Disc Model. FIG. 5A depicts an example of tissue in the vicinity of a site of incision at 2 weeks following surgery. The disrupted collagen architecture, new blood vessels, and cellular infiltrate in the tissue may be discerned. FIG. 5B depicts unperturbed annular tissue, where the distinct lamellae in the annulus may be noted. FIG. 6 depicts an example of the proliferation in annular cells from an incised inter-vertebral disc, as assessed by BrdU staining.

Following the experiment described in Example 3, the extent of inter-vertebral disc degeneration is assessed using the methods described above. Annulus closure tools of the present invention are found to decrease inter-vertebral disc degeneration.

The studies described in the above Examples demonstrate that methods and compositions of the present invention improves healing of annular incisions.

1. A method of sealing an inter-vertebral disk incision, comprising applying an annulus closure tool to an inter-vertebral disk incision, said annulus closure tool comprising a first leg, a second leg, and a connection member connecting said first leg to said second leg, said first leg and said second leg comprising projections protruding therefrom, thereby sealing an inter-vertebral disk incision.

2. The method of claim 1, wherein said inter-vertebral disk incision is a slit incision.

3. The method of claim 1, wherein said annulus closure tool comprises a bioresorbable material.

4. The method of claim 3, wherein said bioresorbable material is a polymer of lactide, glycolide, poly-dioxanone, trimethylene carbonate, polyethylene oxide, poly (epsilon-caprolactone), levolactic acid, or a blend thereof.

5. The method of claim 3, wherein said bioresorbable material is Poly(lactic acid).

6. The method of claim 1, wherein said first leg and said second leg are between about 5 and about 15 mm long.

7. The method of claim 1, wherein said connection member is between about 5 and about 25 mm long.

8. The method of claim 1, wherein said connection member comprises a flexible material.

9. The method of claim 8, wherein said flexible material is a lactide/glycolide copolymer.

10. A method of facilitating healing of an inter-vertebral disk incision, comprising applying an annulus closure tool to said inter-vertebral disk incision, said annulus closure tool comprising a first leg, a second leg, and a connection member connecting said first leg to said second leg, said first leg and said second leg comprising projections protruding therefrom, thereby facilitating healing of an inter-vertebral disk incision.

11. The method of claim 10, wherein said inter-vertebral disk incision is a slit incision.

12. The method of claim 10, wherein said annulus closure tool comprises a bioresorbable material.

13. The method of claim 12, wherein said bioresorbable material is a polymer of lactide, glycolide, poly-dioxanone, trimethylene carbonate, polyethylene oxide, poly (epsilon-caprolactone), levolactic acid or a blend thereof.

14. The method of claim 12, wherein said bioresorbable material is Poly(lactic acid).

15. The method of claim 10, wherein said first leg and said second leg are between about 5 and about 15 mm long.

16. The method of claim 10, wherein said connection member is between about 5 and about 25 mm long.

17. The method of claim 10, wherein said connection member comprises a flexible material.

18. The method of claim 17, wherein said flexible material is a lactide/glycolide copolymer.

19. A method of inhibiting extrusion or re-herniation of a nucleus pulposus, comprising applying an annulus closure tool to an inter-vertebral disk incision from an inter-vertebral disk, wherein the inter-vertebral disk has a tear, fissure, or incision, said annulus closure tool comprising a first leg, a second leg, and a connection member connecting said first leg to said second leg, said first leg and said second leg comprising projections protruding therefrom, thereby inhibiting extrusion or re-herniation of a nucleus pulposus from an inter-vertebral disk.

20. The method of claim 19, wherein said inter-vertebral disk incision is a slit incision.

21. The method of claim 19, wherein said annulus closure tool comprises a bioresorbable material.

22. The method of claim 21, wherein said bioresorbable material is a polymer of lactide, glycolide, poly-dioxanone, trimethylene carbonate, polyethylene oxide, poly (epsilon-caprolactone), levolactic acid, or a blend thereof.

23. The method of claim 21, wherein said bioresorbable material is Poly(lactic acid).

24. The method of claim 19, wherein said first leg and said second leg are between about 5 and about 15 mm long.

25. The method of claim 19, wherein said connection member is between about 5 and about 25 mm long.

26. The method of claim 19, wherein said connection member comprises a flexible material.

27. The method of claim 26, wherein said flexible material is a lactide/glycolide copolymer.

28. A method of inhibiting extrusion or re-herniation of a nucleus replacement device or material from an inter-vertebral disk, comprising applying an annulus closure tool to an inter-vertebral disk incision, said annulus closure tool comprising a first leg, a second leg, and a connection member connecting said first leg to said second leg, said first leg and said second leg comprising projections protruding therefrom, thereby inhibiting extrusion or re-herniation of a nucleus replacement device or material from an inter-vertebral disk.

29. The method of claim 28, wherein said inter-vertebral disk incision is a slit incision.

30. The method of claim 78, wherein said annulus closure tool comprises a bioresorbable material.

31. The method of claim 30, wherein said bioresorbable material is a polymer of lactide, glycolide, poly-dioxanone, trimethylene carbonate, polyethylene oxide, poly (epsilon-caprolactone), levolactic acid, or a blend thereof.

32. The method of claim 30, wherein said bioresorbable material is Poly(lactic acid).

33. The method of claim 28, wherein said first leg and said second leg are between about 5 and about 15 mm long.

34. The method of claim 28, wherein said connection member is between about 5 and about 25 mm long.

35. The method of claim 28, wherein said connection member comprises a flexible material.

36. The method of claim 35, wherein said flexible material is a lactide/glycolide copolymer.
